(19) United States
(12) Patent Application Publication
(10) Pub. No.: US 2013/0245732 A1
(43) Pub. Date: Sep. 19, 2013

(54) LEAD HEADER AND MANUFACTURE THEREOF

(71) Applicants: Per E. Jarl, Jarfalla (SE); Stefan Christiander, Nacka (SE)

(72) Inventors: Per E. Jarl, Jarfalla (SE); Stefan Christiander, Nacka (SE)

(73) Assignee: ST. JUDE MEDICAL AB, Jarfalla (SE)

(21) Appl. No.: 13/422,898

(22) Filed: Mar. 16, 2012

(51) Int. Cl. A61N 1/05; B21D 35/00; B21D 51/16

(52) U.S. Cl. CPC A61N 1/05; B21D 51/16; B21D 35/001
USPC 607/116; 72/324; 29/527.1; 29/428

(57) ABSTRACT

A lead header for an implantable medical lead is in the form of a metal sheet bent to form a metal tube having a lumen. The metal sheet has a protruding portion that extends radially inwardly into the lumen. The protruding portion may be lip arranged in connection with one of the longitudinal sides of the metal sheet that is bent to protrude radially inwardly into the lumen, or it may be a dent formed in the metal sheet. In either case, the protruding portion is configured to transform a rotation of a helical fixation element of the medial lead that is at least partly present in the lumen into a longitudinal movement of the helical fixation element relative to the lead header.

### TECHNICAL FIELD

The present embodiments generally relate to lead headers of implantable medical leads and to methods of manufacturing such lead headers.

### BACKGROUND

Implantable medical leads form the electrical connection between an implantable medical device (IMD), such as cardiac pacemaker, cardiac defibrillator or cardioverter, and body tissue, such as the heart, which is to be electrically stimulated and/or sensed. As is well known, the implantable medical leads connecting the IMD with the tissue may be used for pacing/defibrillation and/or for sensing electrical signals produced by the tissue.

The implantable medical leads of today and in particular cardiac implantable medical leads can generally be divided into two classes depending on the tissue anchoring arrangement of the leads. Firstly, so-called passive fixation leads comprise radially protruding elements in the distal lead ends. These elements can become embedded in the trabecular network inside the heart and thereby provide an anchoring of the lead to the heart tissue. Examples of such protruding elements include collars, tines and fines. Passive leads are generally characterized by low chronic capture threshold and high impedance.

The other class of leads includes so-called active fixation leads. Such a lead typically comprises, in its distal end, a helical fixation element that can be screwed into the endocardium and myocardium to provide the necessary lead-to-tissue anchoring. Generally, active leads have superior ability to fixate without the need for any trabecular network.

The helical fixation element is generally extendable and retractable relative to the distal lead end by providing a post projecting radially inwardly from the lead header. By then applying a rotating motion at a connector pin in the proximal end of the lead, the rotational movement is transferred via an inner conductor and helix shaft up to the helical fixation element. The rotation of the helical fixation element forces, due to the post, the helical fixation element to advance or retract within the lead header.

FIG. 1 is a cross-sectional view of a current design of a typical lead header 100 for an active fixation lead. The lead header 100 basically consists of two separate pieces: a header body 110 and a marker ring 140. These two pieces 110, 140 are typically manufactured as respective out-of-precision drawn tubes and cut to desired length using, for instance, a Swiss machine. The marker ring 140 additionally comprises slots 150 that are used to securely attach a silicon soft tip 170 (see FIG. 2) to the marker ring 140. Correspondingly, the header body 110 comprises a through-hole 120 into which the above mentioned post 160 is inserted during assembling. The header body 110 further comprises a cut out 130 onto which the marker ring 140 is threaded during manufacture. The slots 150, the through-hole 120 and the cut out 130 need to be laser cut prior to assembling the marker ring 140 and the header body 110 together. Laser welding is then applied to securely attach the marker ring 140 to the header body 110.

The post 160 is inserted in the through-hole 120 of the header body 110 and is laser welded to the header body 110. This is a high-precision step due to the very small size of the components. Then the silicon soft tip 170 is molded over the marker ring 140 and a portion of the header body 110 as shown in FIG. 2.

Thus, the manufacture of the lead header 100 for prior art active fixation leads requires high precision and is very complex due to the small size of the components. Hence, the assembling process is costly and requires high skill of the operator that manually assemblies and attaches the different pieces together to form the final lead header 100.

There is therefore a need for a lead header design that simplifies the manufacture process for active fixation leads as compared to prior art solutions.

### SUMMARY

It is a general objective to provide a lead header for implantable medical leads that can be easily manufactured.

An aspect of the embodiments relates to a lead header for an implantable medical lead. The lead header is in the form of a metal sheet bent to form a metal tube having a lumen. The metal sheet has a protruding portion, e.g., lip, arranged in connection with a first longitudinal side of the metal sheet. The lip is bent to protrude radially inwardly into the lumen and is configured to transform a rotation of a helical fixation element at least partly present in the lumen into a longitudinal movement of the helical fixation element relative to the lead header.

Another aspect of the embodiments relates to an implantable medical lead comprising a proximal lead portion connectable to an implantable medical device. A tubular lead body has a first end connected to the proximal lead portion and a second, opposite end connected to a distal lead portion. This distal lead portion comprises a lead header in the form of a metal sheet bent to form a metal tube having a lumen. The metal sheet has a lip arranged in connection with a first longitudinal side and wherein the lip is bent to protrude radially inwardly into the lumen. The distal lead portion also comprises a helical fixation element at least partly present in the lumen of the metal tube. The lip is thereby configured to transform a rotation of the helical fixation element into a longitudinal movement of the helical fixation element relative to the lead header.

A further aspect of the embodiments relates to a method of manufacturing a lead header for an implantable medical device. The method comprises cutting a lip in connection with a first longitudinal side of a metal sheet. The lip is bent in a next step followed by bending the metal sheet to form a metal tube having a lumen. The lip thereby protrudes radially inwardly into the lumen and is configured to transform a rotation of a helical fixation element into a longitudinal movement of the helical fixation element relative to the lead header.

Yet another aspect of the embodiments relates to a lead header for an implantable medical lead. The lead header is in the form of a metal sheet bent to form a metal tube having a lumen. The metal sheet comprises a protruding portion, e.g., dent, protruding radially inwardly into the lumen and is configured to transform a rotation of a helical fixation element at least partly present in the lumen into a longitudinal movement of the helical fixation element relative to the lead header.

A further aspect of the embodiments relates to an implantable medical lead comprising a proximal lead portion connectable to an implantable medical device. A tubular lead body has a first end connected to the proximal lead portion and a second, opposite end connected to a distal lead portion. This distal lead portion comprises a lead header in the form of a metal sheet bent to form a metal tube having a lumen. The metal sheet comprises a dent protruding radially inwardly into the lumen. The distal lead portion also comprises a helical fixation element at least partly present in the lumen of the metal tube. The dent is thereby configured to transform a rotation of the helical fixation element into a longitudinal movement of the helical fixation element relative to the lead header.

Still further aspect of the embodiments relates to a method of manufacturing a lead header for an implantable medical device. The method comprises forming a dent in a metal sheet. The metal sheet is then bent to form a metal tube having a lumen. The dent thereby protrudes radially inwardly into the lumen and is configured to transform a rotation of a helical fixation element into a longitudinal movement of the helical fixation element relative to the lead header.

The lead header of the embodiments is easily manufactured and does not require the handling of a separate post but rather uses an integrated lip or a dent to achieve a rotation-to-translation transformation for the helical fixation element. The components for the lead header can be manufactured in a fully automated or at least semi-automated process, thereby reducing the time and cost for manufacturing the lead header.

### BRIEF DESCRIPTION OF THE DRAWINGS

The invention, together with further objects and advantages thereof, may best be understood by making reference to the following description taken together with the accompanying drawings, in which:

FIG. 1 is a cross-sectional view of a lead header according to prior art;

FIG. 2 is a cross-sectional view of the lead header in FIG. 1 with attached post and silicone soft tip;

FIG. 3 is a schematic drawing of a metal sheet used for forming a lead header according to an embodiment;

FIG. 4 is a cross-sectional view of a lead header according to an embodiment;

FIG. 5 is a cross-sectional view of a lead header according to an embodiment taken along the line A-A in FIG. 4;

FIG. 6 is a schematic drawing of a metal sheet used for forming a lead header according to another embodiment;

FIG. 7 is a schematic drawing of a radiopaque metal sheet used for forming a marker ring according to an embodiment;

FIG. 8 is a cross-sectional view of a lead header with an attached marker ring according to an embodiment;

FIG. 9 is a cross-sectional view of a marker ring according to an embodiment;

FIG. 10 is a cross-sectional view of a lead header with an attached marker ring and a polymer tip tube according to an embodiment;

FIG. 11 is schematic drawing of a metal sheet used for forming a lead header according to a further embodiment;

FIG. 12 is a cross-sectional view of a lead header according to an embodiment taken along the line B-B in FIG. 11 following bending the metal sheet into a tube;

FIG. 13 schematically illustrates an implantable medical lead according to an embodiment connectable to an implantable medical device;

FIG. 14 is an illustration of an implantable medical lead according to an embodiment;

FIG. 15 is a cross-sectional view of a distal portion of an implantable medical lead according to an embodiment;

FIG. 16 is a flow diagram illustrating a method of manufacturing a lead header according to an embodiment;

FIG. 17 is a flow diagram illustrating additional steps of the method in FIG. 16;

FIG. 18 is a flow diagram illustrating an embodiment of attaching the marker ring in FIG. 17;

FIG. 19 illustrates an example of a manufacturing process for a lead header; and

FIG. 20 is a flow diagram illustrating a method of manufacturing a lead header according to another embodiment.

### DETAILED DESCRIPTION

Throughout the drawings, the same reference numbers are used for similar or corresponding elements.

The present embodiments generally relate to lead headers for implantable medical leads and to methods of manufacturing such lead headers. The embodiments are in particular directed towards such lead headers that are used in so-called active fixation leads having a helical fixation element or screw structure that is used to anchor the implantable medical lead in a target tissue.

The lead headers of active fixation leads generally have a post or projecting structure protruding inwardly into a lumen or channel defined by the lead header. There the post is interposed between adjacent turns of the helical fixation element and will transform rotation of the helical fixation element into a longitudinal movement of the helical fixation element relative to the lead header.

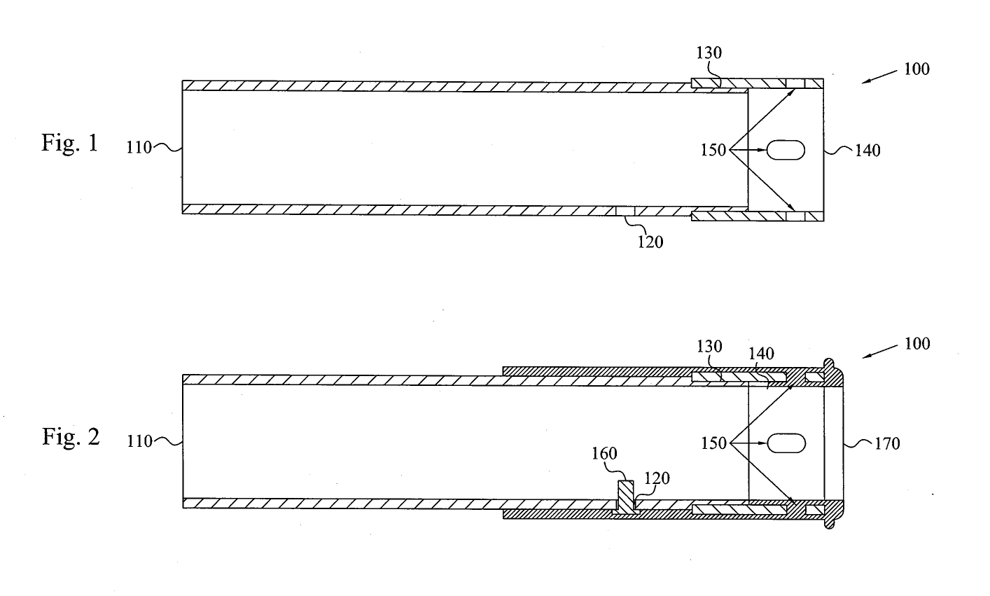

The minute diameter of implantable medical leads and the lead headers of such implantable medical leads make the assembling process very time consuming and skill requiring, in particular with regard to handling the lead header and the post, which has been discussed in the background section in connection with FIGS. 1 and 2.

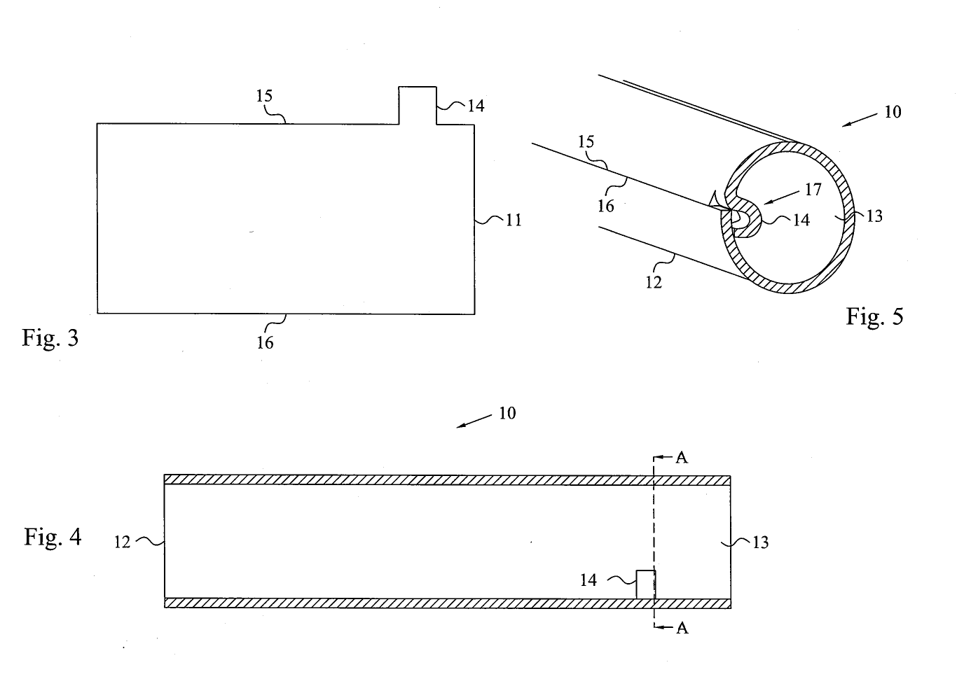

The present embodiments have taken a radically different approach as compared to the prior art by using a lead header in the form of a metal sheet or plate that is bent or folded to form a metal tube or cylinder. FIG. 3 schematically illustrates an embodiment of such a metal sheet 11. The metal sheet 11 has a length corresponding to the length of the lead header in the implantable medical lead and a width that substantially corresponds to the circumference of the metal tube following bending of the metal sheet 11 into a metal tube. In some embodiments, the width of the metal sheet 11 could be somewhat larger than the circumference of the metal tube in the case of a partial overlap of the longitudinal sides 15, 16 of the metal sheet 11 in the joint of the metal tube.

The metal sheet 11 comprises an integrated lip or bendable structure 14 that is arranged in connection with a first longitudinal side 15 of the metal sheet 11. In the embodiment shown in FIG. 3, the lip 14 is arranged at and extends beyond the first longitudinal side 15. This lip 14 is configured to be bent to protrude radially inwardly into the lumen of the metal tube. FIG. 4 illustrates a cross-sectional view of the lead header 10 that is in the form of a metal tube 12 formed by bending the metal sheet 11 of FIG. 3 and by bending the lip 14 to protrude into the lumen 13 of the metal tube 12. FIG. 5 is a cross-sectional view of the metal tube 12 taken along the line A-A shown in FIG. 4. As is clearly seen in FIG. 5, bending the metal sheet 11 into a cylinder will bring the two longitudinal sides 15, 16 close together to form a joint and thereby a metal tube 12 with the lip 14 protruding inwards into the lumen 13 of the metal tube 12.

The lip 14 of the metal tube 12 protruding into the lumen 13 is configured to transform a rotation of a helical fixation element to be at least partly present in the lumen 13 into a longitudinal movement of the helical fixation element relative to the lead header 10. Hence, the lip 14 is then interposed between adjacent turns of the helical fixation element to achieve this rotation-to-translation transformation.

In a particular embodiment, the lip 14 is simply bent to protrude as a straight structure (not shown) into the lumen 13. This generally works well and the width of the lip 14 is thereby selected to be equal to or preferably smaller then the distance between adjacent turns in the helical fixation element in order to be interposed between such adjacent turns.

In this embodiment the lip 14 is basically bent 90 degrees relative to the flat metal sheet 11 in FIG. 3 prior to bending the metal sheet 11 into a tube or cylinder shape. This lip 14 will thereby project radially inwardly into the lumen 13. Also other overall bending configurations of the lip 14 are possible and within the scope of the embodiments. For instance and as shown in FIG. 5, the lip 14 could be bent to a general U-shape to form a post 17 protruding radially inwardly into the lumen 13. Such a U-shaped configuration of the lip 14 might achieve a more stable post structure as compared to a lip 14 bent to form a straight structure protruding into the lumen 13. Hence, the U-shaped lip 14 might withstand any forces exerted by the helical fixation element during rotation of the helical fixation element better without the risk of a structural deformation of the lip 14.

In a particular embodiment the two longitudinal sides 15, 16 of the metal sheet 11 meet each other at a joint following bending the metal sheet 11 into the metal tube 12. In such a case, a weld, such as a laser weld, can be applied between the first longitudinal side 15 and a second, opposite longitudinal side 16 to form a mechanical joint between the longitudinal sides 15, 16. Welding additionally achieves a closed system for the metal tube 12 with regard to its envelope or side surface.

Also other forms of mechanical joints are possible and do not necessarily have to achieve a closed joint. Such mechanical joints could be based on the principles of dovetail joints where a series of pins extending from one of the longitudinal sides 15 interlock with a series of tails in the other longitudinal side 16.

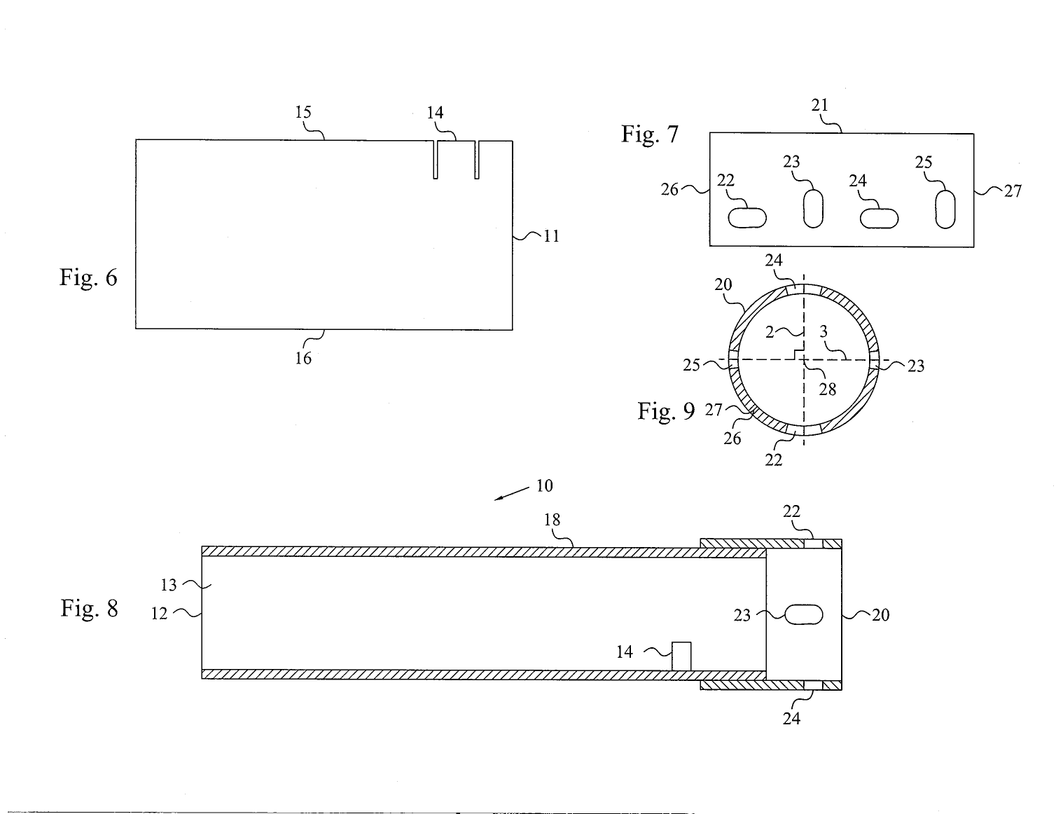

FIG. 6 schematically illustrates another embodiment of a metal sheet 11 that can be bent to form metal tube 12 of a lead header 10 for an implantable medical lead. In this embodiment the lip 14 is arranged in connection with the first longitudinal side 15 but its end is substantially aligned with the first longitudinal side 15. Hence, the lip 14 does not necessarily extend beyond the first longitudinal side 15. The embodiments also encompass any lip structure that is basically an intermediate of the ones shown in FIGS. 3 and 6. Thus, the lip 14 could be attached to the metal sheet body at a position further to the center in the transversal direction of the metal sheet 11 as compared to the first longitudinal side 15, i.e. as shown in FIG. 6. However, the lip 14 may still extend beyond the first longitudinal side 15, i.e. as shown in FIG. 3.

Regardless of arrangement position in connection with the first longitudinal side 15, the lip 14 is bent to protrude radially inwardly into the lumen 13 formed by the metal sheet 11 when it has been bent to form the metal tube 12.

In FIG. 3, the lip 14 is formed by cutting away metal sheet material beyond the first longitudinal side 15, such as by punching, but keeping part of the metal sheet material corresponding to the lip 14 left. In FIG. 6 slots are cut in the metal sheet 11 on either side of the lip 14 unless the lip 14 is arranged in connection with one of the ends of the first longitudinal side 15. In this latter case a single slot is cut or punched in the metal sheet 11 to form the lip 14.

The position of the lip 14 along the first longitudinal side 15 can be selected by the manufacturer to be anywhere from a first end of the first longitudinal side 15 up to the second, opposite end of the first longitudinal side 15.

The metal sheet 11 can be made of any metal material that can be manufactured into a metal sheet 11 and bent to form a metal tube. The metal material should furthermore be non-toxic and implantable in a human or animal body. Non-limiting but preferred examples of such metal materials include titanium, titanium alloys and MP35N®, which is a nickel-cobalt-chromium-molybdenum alloy. It is further preferred if the metal material can be welded to form a joint between the longitudinal sides 15, 16.

In a particular embodiment the lead header also comprises a marker ring in addition to the metal tube formed from the metal sheet. The marker ring is then made of a radiopaque material to be visible through X-ray imaging, for instance during implantation of the implantable medical lead. The radiopaque marker ring that is present close to the distal end and tip of the implantable medical lead will therefore be a tool used by the physician to visually track the tip of the implantable medical lead in the human or animal body during implantation.

The marker ring 20 is attached to a portion of an outer surface 18 (envelope surface) of the metal tube 12 as is shown in FIG. 8. In more detail, the marker ring 20 is attached to the outer surface 18 in connection with a first end of the metal tube 12 and where this first end faces the distal end or tip of the implantable medical lead, into which the lead header 10 is to be assembled.

In an embodiment, the marker ring 20 can be in the form of a drawn tube or ring of the radiopaque material that has an inner diameter that substantially corresponds to the outer diameter of the metal tube 12. The marker ring 20 is then threaded on the metal tube 12 as is shown in FIG. 8.

In another embodiment, the marker ring 20 is in the form of a radiopaque metal sheet 21, see FIG. 7, which is bent or folded to form the marker ring 20. FIG. 7 illustrates an embodiment of such a radiopaque metal sheet 21 and FIG. 9 illustrates a cross-sectional view of the marker ring 20 once the radiopaque metal sheet 21 has been bent to a ring shape with the opposite sides 26, 27 of the radiopaque metal sheet 21 joined as shown in FIG. 9.

In such an embodiment, the radiopaque metal sheet 21 can be placed on a part of the outer surface 18 of the metal tube 12 and then bent around the circumference of the metal tube 12 to form the marker ring 20 of FIG. 8 attached to a portion of the outer surface 18 of the metal tube 12. Alternatively, the radiopaque metal sheet 21 is first bent to form the marker ring 20, which is then threaded on the metal tube 12.

The first side 26 of the radiopaque metal sheet 21 can be joined to the second, opposite side 27 by any of the joining techniques disclosed in the foregoing for the metal sheet 11. For instance, welding, such as laser welding, can be applied to form a closed joint. Also other mechanical joining techniques can be used, such as a dovetail joint.

In a particular embodiment, a joint is further applied between a portion of the inner surface of the marker ring 20 and a portion of the outer surface 18 of the metal tube 12. Such a joint could be a circumferential weld, such as a circumferential laser weld, to obtain a closed joint. Other types of joint and welding techniques can be used.

In an embodiment, the radiopaque metal sheet 21 comprises at least two slotted through-holes 22, 23, 24, 25 angled relative to each other. These through-holes 22, 23, 24, 25 can then be used to attach a polymer tip tube to the marker ring 20, which is further disclosed herein. The relative angles of the through-holes is thought to improve the connection between the marker ring 20 and the polymer tip tube and prevent the polymer tip tube from unintentionally falling off the marker ring 20.

The angled through-holes 22, 23, 24, 25 have a further benefit in that they enable rotation orientation of the implantable medical lead during implantation through X-ray imaging. Thus, an X-ray image of the implantable medical lead in the human or animal body clearly shows the marker ring and the angled through-holes 22, 23, 24, 25 therein. This will guide the physician and provide rotation information that can be used if a particular lead rotation orientation is desired.

The angled through-holes 22, 23, 24, 25 are preferably positioned on the radiopaque metal sheet 21 so that a first straight connection line 2 between a center 28 of the marker ring 20 and a center of a first through-hole 22, 24 is perpendicular to a second straight connection line 3 between the center 28 of the marker ring 20 and a center of a second through-hole 23, 25. This is shown in FIGS. 8 and 9. In a particular embodiment the radiopaque metal sheet 21 comprises four slotted through-holes 22, 23, 24, 25 angled about 90 degrees relative to each other and positioned so that the first straight connection line 2 between the center of the first through-hole 22, the center 28 of the marker ring 20 and a center of a third through-hole 24 is perpendicular to the second straight connection line 3 between the center of the second through-hole 23, the center 28 of the marker ring 20 and a center of a fourth through-hole 25.

In this embodiment, the through-holes 22, 23, 24, 25 have different dimensions with the first and third through-holes 22, 24 having larger lengths as compared to their widths while the second and fourth through-holes 23, 25 have larger widths as compared to their lengths.

The marker ring 20 can be made of any radiopaque material that can be visually seen in a human or animal body through X-ray imaging and that is non-toxic and implantable. The radiopaque material is preferably selected so that it can be manufactured into a radiopaque metal sheet 21 and bent to form the marker ring 20. Non-limiting but preferred examples of such radiopaque materials include radiopaque metal materials selected from tantalum and alloys of platinum and iridium. It is further preferred if the radiopaque material can be welded to form a joint between the sides 26, 27 and furthermore is weldable to the metal material of the metal sheet 11. Tantalum is generally readily weldable to titanium materials and a platinum-iridium alloy is typically weldable to MP35N®.

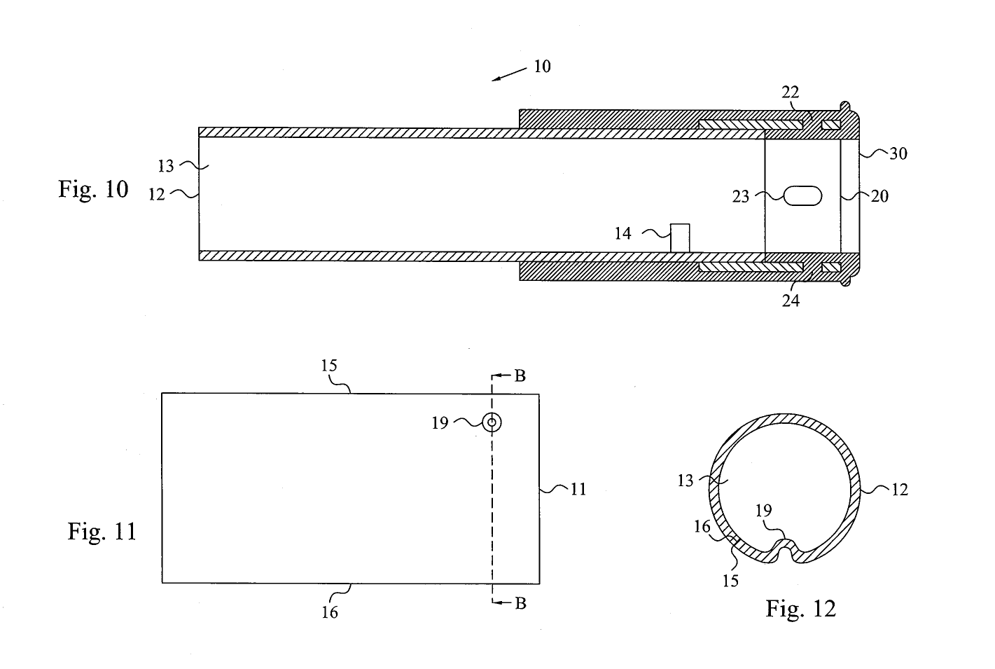

FIG. 10 is a cross-sectional view of an embodiment of a lead header 10 with the metal tube 12 and its bent lip 14, the marker ring 20 and a polymer tip tube 30 molded on the marker ring 20 and at least a portion of the metal tube 12. The polymer tip tube 30 can be made of any electrically isolating polymer material that can be molded over the marker ring 20 and the metal tube 12. A non-limiting example of suitable polymer material is silicone.

The through-holes 22, 23, 24 of the marker ring 20 enables the polymer material to penetrate from the outside of the marker ring 20 through the through-holes 22, 23, 24 and extend along the inner surface of the marker ring 20 as is shown in FIG. 10. The polymer tip tube 30 is therefore securely anchored in the marker ring 20 and is thereby prevented from unintentionally falling off the lead header 10.

In the embodiments discussed in the foregoing the metal sheet that is formed to the metal tube of the lead header comprises a bendable integrated lip that is cut or punched in the metal sheet to be arranged in connection with the first longitudinal side of the metal sheet. FIGS. 11 and 12 illustrate another embodiment of achieving a post structure from a metal sheet. In this embodiment a dent 19 is formed in the metal sheet 11 by applying pressure to one of the main surfaces of the metal sheet 11, for instance by punching. The dent 19 will then project along a normal to the main surface of the metal sheet 11. This means that when the metal sheet 11 is bent to form the metal tube 12 the dent 19 will protrude radially inwardly into the lumen 13 of the metal tube. FIG. 12 illustrates a cross-sectional view taken along the line B-B in FIG. 11 following bending the metal sheet 11 into a metal tube 12. The dent 19 will thereby be interposed between adjacent turns of a helical fixation element at least partly present in the lumen 13. The dent 19 transforms a rotation of the helical fixation element to a longitudinal movement of the helical fixation element relative to the lead header and the metal tube 12.

The embodiment of the metal sheet 11 and metal tube 12 shown in FIGS. 11 and 12 can be used together with the marker ring and polymer tip tube discussed in the foregoing and shown in FIGS. 7 to 10.

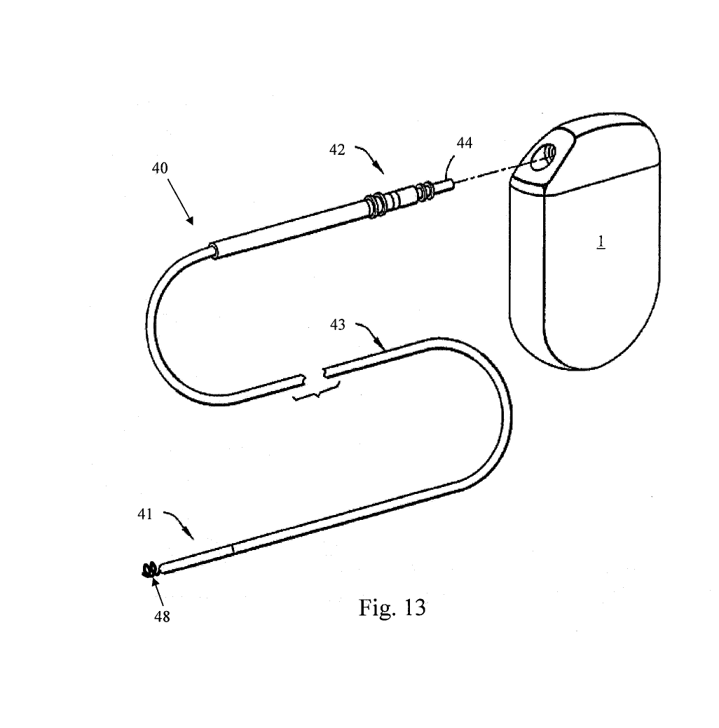

FIG. 13 is a schematic overview of an implantable medical lead 40 according to an embodiment comprising a lead header as disclosed herein. The implantable medical lead 40 basically comprises a proximal lead portion 42 that is connectable to an implantable medical device 1, such as a pacemaker, a cardiac defibrillator or cardioverter. The proximal lead portion 42 is connected to a first end of a tubular lead body 43 having its second, opposite end connected to a distal lead portion 41 comprising the lead header and the helical fixation element 48.

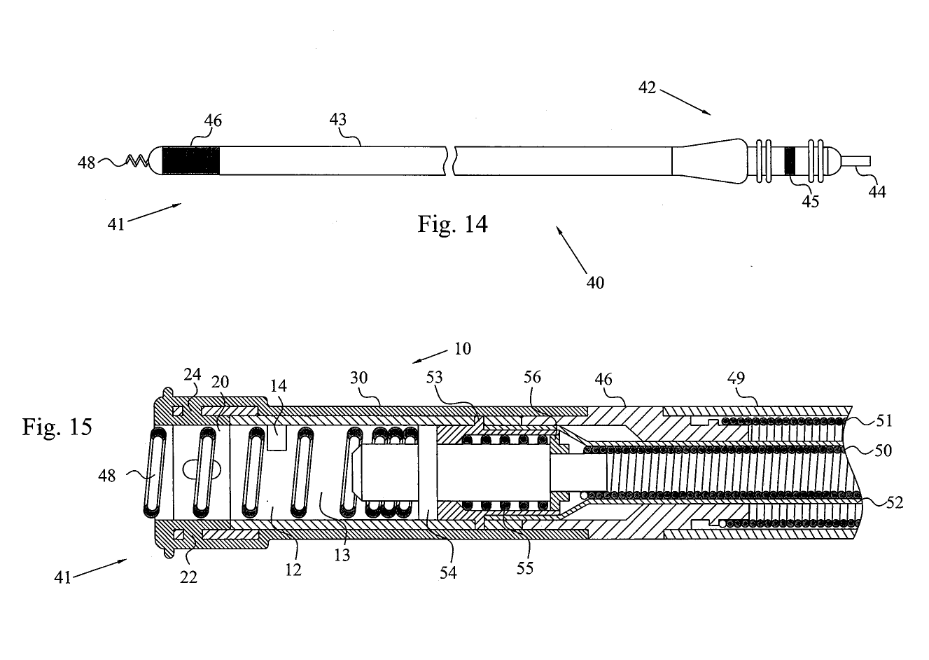

FIG. 14 illustrates the implantable medical lead 40 in more detail. The proximal lead portion 42 preferably comprises a rotatable connector 44, also sometimes referred to as a connector pin. The rotatable connector 44 is connectable to the implantable medical device. The rotatable connector 44 is mechanically and electrically connected to a conductor, typically in the form of a conductor coil running in a lumen of the tubular lead body 43. The opposite end of the conductor is mechanically and electrically connected to the helical fixation element 48. Thus, when the physician would like to move the helical fixation element 48 out from the lead header in the distal lead portion 41 or retract the helical fixation element 48 back into the lead header, he/she rotates the rotatable connector 44. This rotation is propagated through the conductor down to the helical fixation element 48. The lip or dent present in the metal tube of the lead header projecting into the lumen and interposed between adjacent turns of the helical fixation element 48 transforms this rotation into a longitudinal movement of the helical fixation element 48 relative to the lead header.

The implantable medical lead 40 could be a so-called bipolar or multipolar lead. In such a case, it comprises at least one electrode 46 in the distal lead portion 41 in addition to the helical fixation electrode 48 that preferably operates as a sensing and/or pacing electrode. The at least one other electrode 46 is then typically a so-called ring electrode 46 that is electrically connected to a second conductor, such as an outer conductor coil, running in the lumen of the tubular lead body 43 and ends at a ring connector 45 in the proximal lead portion 42.

FIG. 15 is a cross-sectional view of the distal portion 41 of the implantable medical lead showing the lead header 10 of the embodiments. FIG. 15 illustrates that the helical fixation element 48 is mechanically and electrically connected to an inner conductor coil 50 by means of the helix shaft 54 manufactured by an electrically conductive material such as platinum, gold, tantalum, titanium or an alloy such as platinum/iridium, e.g. Pt/Ir 90/10 or 80/20. The lead-connecting (proximal) end of the helical fixation element 48 and the distal end of the inner conductor coil 50 may be attached by, for instance laser welding or the like, to the opposite ends of the helix shaft 54. The helix shaft 54 is journaled for rotation and axial movement within a sleeve 53 and includes a radially extending flange defining a proximal, radially-extending surface engageable against a distal extremity of the sleeve 53 to limit the retraction of the helical fixation element 48. The sleeve 53 is preferably electrically conductive and secured to the metal tube 12 of the lead header 10.

The proximal portion of the sleeve 53 has a counterbore terminating at a distal end wall. An electrically conductive tubular abutment 56, such as of MP35N® or the like, L-shaped in cross section, has an axial portion connected, e.g. welded, to the proximal end of the helix shaft 54 and a flange projecting radially within the counterbore of the sleeve 53. Thus, the abutment 56 being secured to the helix shaft 54 is movable rotationally and axially with the helix shaft 54 relative to the sleeve 53.

Contained within the counterbore is an expandable/contractable contact member, preferably in the form of a metallic compression spring 55. The compression spring 55 prevents the helical fixation element 48 from unintentionally moving out from the lead header 10 during implantation.

An outer insulating tube 49, for instance of silicone rubber, polyurethane or OPTIM®, extends proximally from the ring electrode 46 and covers the main lead body up to the proximal lead portion.

The ring electrode 46 is in electrical contact with the ring connector of the proximal lead portion through an outer conductor coil 51. The two conductor coils 50, 51 are electrically insulated by a longitudinally extending insulating tube 52, such as made of silicone rubber, polyurethane or the like. The insulting tube 52 is disposed between the conductor coils 50, 51 to prevent electrical contact between the conductors 50, 51 and between the ring electrode 46 and the inner conductor coil 50.

The distal lead portion 41 additionally comprises the lead header 10 with the metal tube 12, the marker ring 20 and the polymer tip tube 30 previously discussed herein. FIG. 15 clearly illustrates how the helical fixation element 48 is at least partly present in the lumen 13 of the metal tube 12 so that the lip 14 or the dent protrudes radially inwardly into the lumen 13 and is interposed between adjacent turns of the helical fixation element 48.

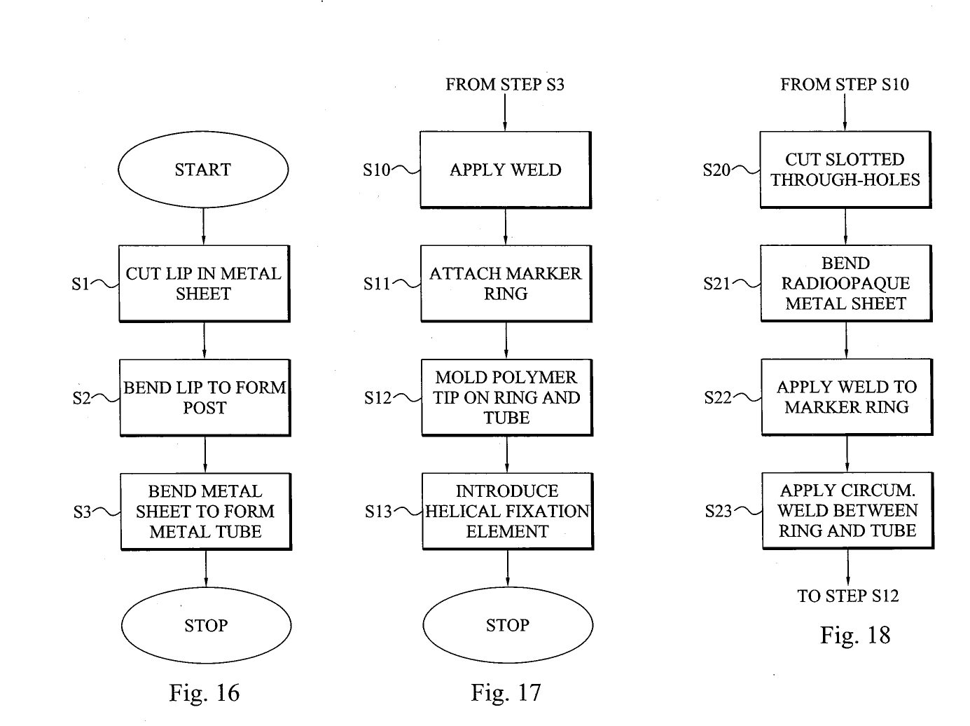

FIG. 16 is a flow diagram of a method for manufacturing a lead header for an implantable medical lead according to an embodiment. The method generally starts in step S1 where a lip is cut in connection with a first longitudinal side of a metal sheet. The lip can be formed by cutting or punching away excessive metal material on either sides of a portion of the metal sheet to get a lip extending beyond the first longitudinal side of the metal sheet as shown in FIG. 3. Alternatively, one or more slits can be cut or punched in the metal sheet to get a lip embodiment as shown in FIG. 6.

A next step S2 bends the lip to form a post the projects substantially perpendicular from a main surface of the metal sheet. Hence the lip preferably extends along a normal to this main surface after the bending in step S2. The lip is optionally bent to form a U-shaped post as shown in FIG. 5.

In a next step S3 the metal sheet is bent to form a metal tube having a lumen. By bending the lip in step S2 the lip protrudes radially inwardly into the lumen following step S3 to thereby be configured to transform a rotation of a helical fixation element at least partly present in the lumen into a longitudinal movement of the helical fixation element relative to the lead header.

FIG. 17 is a flow diagram illustrating additional, optional steps of the method in FIG. 16. The method continues from step S3 in FIG. 16. A next step S10 applies a weld, such as a laser weld, between the longitudinal sides of the metal sheet to form a closed joint in the metal tube. Alternative embodiments for joining the longitudinal sides of the metal sheet are possible as previously disclosed herein.

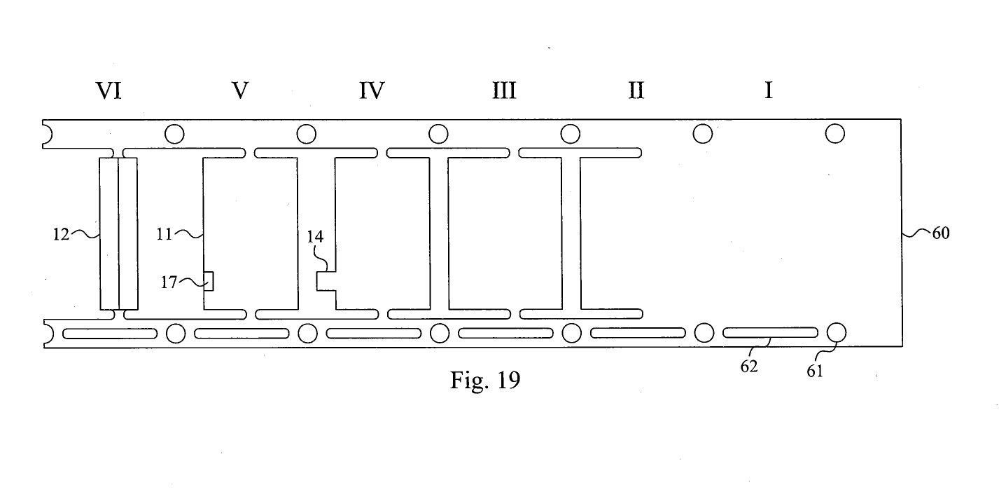

A marker ring of a radiopaque material is attached to a portion of an outer surface of the metal tube in step S11. If the marker ring is in the form of a pre-shaped ring step S11 preferably involves threading the marker ring on the outer surface of the metal tube. Other embodiments of attaching the marker ring will be discussed further in connection with FIG. 18 below.

Step S12 molds a polymer tip tube on the marker ring and at least a portion of the metal tube. Thereafter the helical fixation element is introduced into the lumen of the metal tube in step S13 to thereby have the lip or dent in the metal tube interposed between adjacent turns of the helical fixation element.

FIG. 18 is a flow diagram illustrating a particular embodiment of attaching the marker ring in FIG. 17. The method continues from step S10 in FIG. 17. A next step S20 cuts or punches slotted through-holes in a radiopaque metal sheet. At least two such slotted through-holes angled relative to each other are preferably cut in the radiopaque metal sheet as previously disclosed herein. The radiopaque metal sheet with the optional but preferred slotted through-holes is then bent to form the marker ring in step S21. Step S21 could bend the radiopaque metal sheet around a portion of the metal tube. Alternatively, the radiopaque metal sheet is first bent to form the marker ring. The marker ring is then threaded onto the portion of the metal tube.

Step S22 applies a weld between opposite sides of the radiopaque metal sheet to form a, preferably closed, joint in the marker ring. Alternatively other mechanical joints as discussed herein could be used. Step S22 is preferably performed after attaching the marker ring to the metal tube but could actually be performed prior to threading the marker ring onto the metal tube.

Step S23 applies a circumferential joint, such as weld and preferably laser weld, between the marker ring and the metal tube to mechanically attach the marker ring to the metal tube. The method then continues to step S12 of FIG. 17 to mold the polymer tip tube on the marker ring and the metal tube.

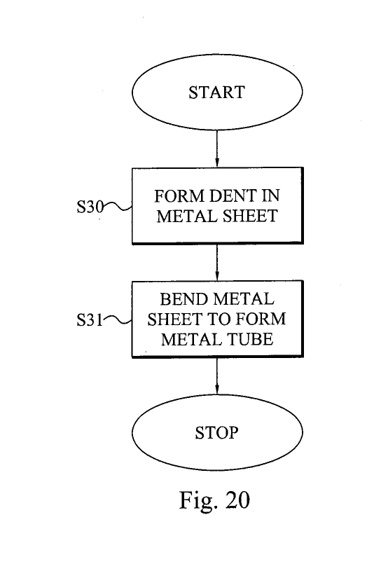

When manufacturing a lead header based on the metal sheet as disclosed in FIGS. 11 and 12, steps S1 and S2 of FIG. 16 are replaced by forming the dent in the metal sheet as shown in step S30 of FIG. 20. This dent is preferably formed by applying pressure to, such as punching, the metal sheet. The method then continues to step S31 to bend the metal sheet with the dent into a metal tube with the dent protruding radially inwardly into the lumen of the metal tube. The optional steps of FIGS. 17 and 18 can also be applied to the method illustrated in FIG. 20.

FIG. 19 schematically illustrates how a metal sheet for the lead header can be manufactured from a metal strip 60 in a serial process. A metal strip 60 is input in the manufacture process and guiding structures 61, 62, represented by through-holes 61 and slotted through-holed 62 in FIG. 19, are cut or punched in the metal strip 60 in sub-process I. These guiding structures 61, 62 are used in the manufacture process to correctly align the metal strip 60 to the different cutting and bending sub-processes II-VI. A next sub-process II cuts one half of the metal sheet 11 with the following sub-process III cutting the other half to form the metal sheet 11 attached to the metal strip 60 at its two short sides. A next sub-process IV cuts the lip 14 in connection with the first longitudinal side of the metal sheet 11. These sub-processes II to IV could be replaced by single cutting or punching sub-process in which the outer contour of the metal sheet with the lip 14 is formed directly from the metal strip 60. Alternatively, the three sub-processes II to IV could be replaced by two sub-processes where sub-process II both cuts half the metal sheet 11 and the lip 14 in a single cutting/punching operation and with sub-process III cutting the other half of the metal sheet 11. Sub-process V bends the lip 14 to form a post extending substantially parallel to a normal of the main surface of the metal sheet 11. Finally sub-process VI bends the metal sheet 11 to form the metal tube 12 with the lid 14/post 17 protruding radially inwardly into the lumen defined by the metal tube 12.

Following sub-processes could apply a weld along the joint between the longitudinal sides of the metal sheet and finally cutting away the metal tube 12 from the metal strip 60.

A similar procedure using a radiopaque metal strip can be used for manufacturing the marker ring of the lead header.

Hence, the lead header of the embodiments can be manufactured in a simple process that can be automated thereby reducing the cost and time of manufacturing and assembling a lead header for an implantable medical lead.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

What is claimed is:

1. A lead header for an implantable medical lead with a helical fixation element, said lead header comprising:
a metal sheet bent to form a metal tube having a lumen; and
a protruding portion of the metal sheet configured to extend radially inwardly into the lumen and to transform a rotation of the helical fixation element at least partly present in the lumen into a longitudinal movement of the helical fixation element relative to the lead header.

2. The lead header of claim 1, wherein the helical fixation element comprises a plurality of adjacent turns separated by a distance and the protruding portion is configured to be interposed between adjacent turns of the helical fixation element.

3. The lead header of claim 1, wherein the protruding portion comprises a lip arranged in connection with a first longitudinal side of the metal sheet and bent to protrude radially inwardly into the lumen.

4. The lead header of claim 3, wherein the lip extends beyond the first longitudinal side of the metal sheet.

5. The lead header of claim 3, wherein the lip is bent to form a U-shaped post protruding radially inwardly into the lumen.

6. The lead header of claim 3, comprising a weld applied between the first longitudinal side of the metal sheet and a second, opposite longitudinal side of the metal sheet.

7. The lead header of claim 1, wherein the protruding portion comprises a dent formed in the metal sheet so as to protrude radially inwardly into the lumen.

8. The lead header of claim 1, comprising a marker ring of a radiopaque material attached to a portion of an outer surface of the metal tube.

9. The lead header of claim 8, wherein the marker ring is in the form of a radiopaque metal sheet bent to form the marker ring.

10. The lead header of claim 9, comprising a weld applied between a first side of the radiopaque metal sheet and a second, opposite side of the radiopaque metal sheet.

11. The lead header of claim 9, wherein the radiopaque metal sheet comprises at least two slotted through-holes angled relative to each other and positioned on the radiopaque metal sheet so that a first straight connection line between a center of the marker ring and a center of a first slotted through-hole of the at least two slotted through-holes is perpendicular to a second straight connection line between the center of the marker ring and a center of a second slotted through-hole of the at least two slotted through-holes.

12. The lead header of claim 11, wherein the radiopaque metal sheet comprises four slotted through-holes angled about 90 degrees relative to each other and positioned on the radiopaque metal sheet so that the first straight connection line between the center of the first slotted through-hole, the center of the marker ring and a center of a third slotted through-hole is perpendicular to the second straight connection line between the center of the second slotted through-hole, the center of the marker ring and a center of a fourth slotted through-hole.

13. The lead header of claim 8, comprising a circumferential weld applied between the metal tube and the marker ring around a circumference of the metal tube and a circumference of the marker ring.

14. The lead header of claim 8, comprising a polymer tip tube molded on the marker ring and at least a portion of the metal tube.

15. An implantable medical lead comprising:
a proximal lead portion connectable to an implantable medical device;
a tubular lead body having a first end connected to the proximal lead portion; and
a distal lead portion connected to a second, opposite end of the tubular lead body, the distal lead portion comprises:
a lead header in the form of a metal sheet bent to form a metal tube having a lumen, the metal sheet having protruding portion configured to extend radially inwardly into the lumen; and
a helical fixation element at least partly present in the lumen of the metal tube, wherein the protruding portion is configured to transform a rotation of the helical fixation element into a longitudinal movement of the helical fixation element relative to the lead header.

16. The lead header of claim 15, wherein the protruding portion comprises a lip arranged in connection with a first longitudinal side of the metal sheet and bent to protrude radially inwardly into the lumen.

17. The lead header of claim 15, wherein the protruding portion comprises a dent formed in the metal sheet so as to protrude radially inwardly into the lumen.

18. The implantable medical lead of claim 15, wherein
the proximal lead portion comprises a rotatable connector connectable to the implantable medical device;
the tubular lead body has a lumen housing a conductor having a first end connected to the rotatable connector;
the helical fixation element is connected to a second, opposite end of the conductor; and
the protruding portion is configured to transform a rotation of the connector, the conductor and the helical fixation element into a longitudinal movement of the helical fixation element relative to the lead header.

19. A method of manufacturing a lead header for an implantable medical lead with a helical fixation element having a plurality of adjacent turns separated by a distance, said method comprising:
forming a protruding portion of a metal sheet, wherein the protruding portion has a dimension less than the distance between adjacent turns of the helical fixation element; and
bending the metal sheet to form a metal tube having a lumen, wherein the protruding portion extends radially inwardly into the lumen.

20. The method of claim 19, wherein forming a protruding portion comprises:
cutting a lip in connection with a first longitudinal side of a metal sheet; and
bending the lip.

21. The method of claim 20, wherein cutting the lip comprises punching the lip extending beyond the first longitudinal side of the metal sheet.

22. The method of claim 20, wherein bending the lip comprises bending the lip to form a U-shaped structure.

23. The method of claim 20, comprising applying a weld between the first longitudinal side of the metal sheet and a second, opposite longitudinal side of the metal sheet.

24. The method of claim 17, wherein forming a protruding portion comprises forming a dent in the metal sheet.

25. The method of claim 19, comprising attaching a marker ring of a radiopaque material to a portion of an outer surface of the metal tube.

26. The method of claim 25, wherein attaching the marker ring comprises bending a radiopaque metal sheet around the outer surface of the metal tube to form the marker ring.

27. The method of claim 26, comprising cutting at least two slotted through-holes angled relative to each other in the radiopaque metal sheet, the at least two slotted through-holes are positioned on the radiopaque metal sheet so that a first straight connection line between a center of the marker ring and a center of a first slotted through-hole of the at least two slotted through-holes is perpendicular to a second straight connection line between the center of the marker ring and a center of a second slotted through-hole of the at least two slotted through-holes.

28. The method of claim 27, wherein cutting the at least two slotted through-holes comprises cutting four slotted through-holes angled about 90 degrees relative to each other in the radiopaque metal sheet, the four slotted through-holes are positioned on the radiopaque metal sheet so that the first straight connection line between the center of the first slotted through-hole, the center of the marker ring and a center of a third slotted through-hole is perpendicular to the second straight connection line between the center of the second slotted through-hole, the center of the marker ring and a center of a fourth slotted through-hole.

29. The method of claim 26, comprising applying a weld between a first side of the radiopaque metal sheet and a second, opposite side of the radiopaque metal sheet.

30. The method of claim 25, comprising applying a circumferential weld between the metal tube and the marker ring around a circumference of the metal tube and a circumference of the marker ring.

31. The method of claim 25, comprising molding a polymer tip tube over the marker ring and at least a portion of the metal tube.
